(19) United States
(12) Patent Application Publication
(10) Pub. No.: US 2020/0297590 A1
(43) Pub. Date: Sep. 24, 2020

(54) COSMETIC COMPOSITION COMPRISING CHOLESTERIC LIQUID CRYSTAL PARTICLE AND METHOD FOR PREPARING THE SAME

(71) Applicant: LG HOUSEHOLD & HEALTH CARE LTD., Seoul (KR)

(72) Inventors: Kyohoon LEE, Daejeon (KR); Seong Eun KIM, Daejeon (KR); Jong Sub HAN, Daejeon (KR)

(73) Assignee: LG HOUSEHOLD & HEALTH CARE LTD., Seoul (KR)

(21) Appl. No.: 16/769,419

(22) PCT Filed: Dec. 6, 2017
(86) PCT No.: PCT/KR2017/014200

§ 371 Date: Jun. 3, 2020

(51) Int. Cl. A61K 8/02; A61K 8/06; A61K 8/34; A61K 8/73

(52) U.S. Cl. CPC A61K 8/0295; A61K 8/062; A61K 8/064; A61K 8/345; A61K 2800/805; A61K 8/737; A61K 2800/48; A61K 2800/10; A61K 8/731

(57) ABSTRACT

The present invention provides a cosmetic composition comprising a cholesteric liquid crystal particle having an average diameter of 50 μm to 500 μm and a method for preparing the same. Since the cosmetic composition according to the present invention comprises a cholesteric liquid crystal particle having a controlled average particle diameter and a narrow particle size distribution, the expression of visual dependence of changing color or color tone according to a viewing angle is enhanced.

### TECHNICAL FIELD

The present invention relates to a cosmetic composition comprising a cholesteric liquid crystal particle and a method for preparing the same, and more specifically relates to a cosmetic composition wherein the size and particle size distribution of a cholesteric liquid crystal particle are controlled, and a viewing angle dependence is enhanced thereby, where color changes according to a viewing angle, which is a unique characteristic of the appearance of cholesteric liquid crystal particles.

### BACKGROUND ART

A cholesteric liquid crystal is a liquid crystal in which each layer of molecules, in which the molecular arrangement is arranged in a plane, rotates in a spiral shape, and is used in temperature sensors, display devices, etc. by utilizing the unique optical properties thereof. In recent years, as one characteristic that a cholesteric liquid crystal has, cosmetics using visual dependence of changing the color tone depending on a viewing angle have attracted attention.

For example, JP2015-063477A discloses an emulsion cosmetic composition comprising a cholesteric liquid crystal particle obtained by pulverizing a light reflecting layer formed by immobilizing at least one of a right pivotal cholesteric liquid crystal phase and a left pivotal cholesteric liquid crystal phase, an alcohol having an alkyl group of 2 to 5 carbon atoms, an emulsion, and an emulsifying agent. JP2010-90206A discloses a liquid crystal composition having a cholesteric phase at room temperature or near body temperature using vegetable raw materials and showing an aesthetic decorative effect when applied to human lips or skin, and a cosmetic composition comprising the composition.

However, it is difficult to produce certain forms of appearance by using a cholesteric liquid crystal in a formulation by the conventional methods disclosed in the above patents, and there is a problem that the aesthetic characteristics of appearance may be lost due to stability problems where the structure of a cholesteric liquid crystal is broken during the manufacturing process.

### DISCLOSURE

### Technical Problem

In this regard, as a result of efforts to contain a cholesteric liquid crystal in a liquid formulation in certain forms, the present inventors completed the present invention by confirming that when a cholesteric liquid crystal particle having a uniform particle size distribution while controlling the particle size by membrane emulsification is dispersed in an aqueous phase to prepare an emulsion, stability of particles can be achieved, and also the characteristic of color change according to a viewing angle, which is a unique characteristic of the appearance of a cholesteric liquid crystal particle, can be enhanced.

### Technical Solution

An object of the present invention is to provide a cosmetic composition wherein the expression of dependence on a viewing angle, in which the color changes according to the viewing angle, which is a unique characteristic of the appearance of a cholesteric liquid crystal, is enhanced.

Another object of the present invention is to provide a method for preparing a cosmetic composition comprising the cholesteric liquid crystal particle.

### Advantageous Effect

Since in the cosmetic composition according to the present invention, which comprises a cholesteric liquid crystal particle, the average diameter of particles is controlled to a range of 50 μm to 500 μm, and since the cosmetic composition comprises a cholesteric liquid crystal particle having a narrow particle size distribution, the visual dependence where color or color tone changes according to a viewing angle is significantly enhanced.

### BRIEF DESCRIPTION OF DRAWINGS

FIG. 1 is a mimetic diagram describing an apparatus to which a method for preparing cosmetics according to the present invention is applied.

FIG. 2 is an image of a cosmetic composition comprising a cholesteric liquid crystal particle prepared according to the preparation method of the present invention.

FIG. 3 is an image of a cosmetic composition comprising a cholesteric liquid crystal particle prepared using a stirrer-type emulsification tank.

FIG. 4 is an image of a cosmetic composition comprising a cholesteric liquid crystal particle prepared using a homogenizer mixer.

FIG. 5 is a graph comparing the size distribution of a composition prepared according to the preparation method of the present invention and a cosmetic composition prepared using a stirrer type and a homogenizer mixer.

### BEST MODE

To achieve the objects above, the cosmetic composition of the present invention comprises a cholesteric liquid crystal particle having an average diameter of 50 μm to 500 μm.

The cholesteric liquid crystal particle may be present in the form of an oil-in-water emulsion or a water-in-oil emulsion.

The cholesteric liquid crystal particle may have a value of the degree of dispersion (a) of 0.35 or less.

The content of the cholesteric liquid crystal particle may be 0.1 wt. % to 10 wt. %.

The emulsion may be an oil-in-water emulsion, and the aqueous phase portion of the oil-in-water emulsion may comprise 10 wt. % to 50 wt. % of a moisturizing agent, 0.01 wt. % to 1.0 wt. % of a thickening agent, and purified water.

The method for preparing a cosmetic composition according to the present invention may comprise preparing a liquid crystal portion comprising a cholesteric liquid crystal, respectively; and passing the liquid crystal portion through a porous membrane and then dispersing into a continuous phase, thereby converting the liquid crystal portion to a liquid crystal particle dispersed phase, and thereby obtaining an emulsion comprising a cholesteric liquid crystal particle.

The preparation method may further comprise cooling an emulsion.

The porous membrane may be porous alumina, porous zirconia, porous stainless steel, or porous glass.

Pores of the porous membrane may have an average diameter of 0.1 μm to 2 mm.

### DETAILED DESCRIPTION

The cosmetic composition according to the present invention comprises a cholesteric liquid crystal particle as a dispersed phase or continuous phase of an oil-in-water emulsion or water-in-oil emulsion.

In the present invention, a cholesteric liquid crystal refers to a compound, the molecular structure of which has a helical structure in the liquid crystal state. These are layered materials and have a structure in which the orientation axis changes from layer to layer. Therefore, when light is irradiated to a cholesteric liquid crystal, light of circular polarization in a specific wavelength region corresponding to the rotation direction of the spiral of the liquid crystal molecules and the length of the pitch is reflected. For example, when visible light is irradiated, it selectively reflects light of a specific wavelength corresponding to the pitch length of the liquid crystal. Additionally, cholesteric liquid crystals have an optical characteristic that, unlike pigments or dyes that exhibit color due to absorption of light, they have visual dependence of changing color tone by a viewing angle, and the present invention is characterized in enhancing the expression degree of such visual dependence.

The cholesteric liquid crystal applicable to the cosmetic composition of the present invention is not particularly limited, and any liquid crystal compound capable of expressing visual dependence of changing color or color tone depending on the viewing angle may be used. For example, the liquid crystal may be cholesterol derived from an animal or plant, derivatives thereof, or the one derived from polymers.

Preferably, considering that the cosmetic composition of the present invention may be in direct contact with the skin of the human body or a part of the composition may be absorbed, cholesterol or a derived compound thereof is used as the liquid crystal. Some cholesterol derivatives, in addition to their properties as liquid crystals, have an excellent skin protection effect due to excellent stability and skin miniaturization as constitutional components of skin cell lipids, and can protect the skin from external irritation without side effects, and it is known that it can be used as a composition for the recovery or prevention of damaged skin (for example, refer to KR2014-0147505A).

The cholesterol derivative refers to a compound obtained by halogenating or esterifying the hydroxyl group of the end of a sterol molecule. The cholesterol or a derivative thereof can be selected from, for example, cholesterol, cholesteryl oleyl carbonate, cholesteryl nanonate, cholesteryl chloride, cholestanol, coprostanol, Campesterol, stigmasterol, cytosterol, ergosterol, cerevisterol, chimosterol, dihydrocholesterol, cerebrosterol, lanosterol, dehydrolanosterol, agnosterol, dihydroagnosterol, phytosterol, etc. This can be used alone or in a combination of two or more. The combination is a means usually performed in the art to increase or decrease the temperature indicating a liquid crystal state.

In the cosmetic composition of the present invention, the cholesteric liquid crystal particle is present in the form of a dispersed phase or continuous phase in an oil-in-water or water-in-oil emulsion. The oil-in-water emulsion is a form in which an oil phase portion including the liquid crystal in the continuous phase of an aqueous phase is present as a dispersed phase in the form of droplets, and the water-in-oil emulsion is the opposite form. When cholesterol or a derivative thereof is used as the cholesteric liquid crystal compound, the former is the normal form, but is not limited thereto.

In the cosmetic composition of the present invention, the size of the cholesteric liquid crystal particles forming the dispersed phase have an average diameter of 50 μm to 500 μm, preferably 50 μm to 300 μm, and more preferably 100 μm to 200 μm. According to the inventors' repeated experiments, it was confirmed that when the cholesteric liquid crystal compound is present in a dispersed phase, visual dependence of changes in the color tone by the viewing angle is affected by the average size of particles. Specifically, if the average diameter does not reach 50 μm, it is suspended and the visual dependence depending on the angle is lost. Meanwhile, when the average diameter exceeds 500 μm, the probability of incorporation between particles increases, which greatly reduces formulation stability.

In addition, the particle size distribution of droplets present in the dispersed phase preferably has a dispersion degree (a) of 0.35 or less (refer to Journal of the Japanese Society for Food Science and Technology, 42, 548±555), and more preferably has a dispersion degree (a) of 0.3 or less. Specifically, when a exceeds 0.35, since the color does not change constantly by angle, the uniformity of the prepared particles is of significant importance.

The physical properties of the particles prepared by the present invention are measured as follows.

1) Average particle diameter: The average particle diameter of particles was measured using a light-scattering particle size analyzer (MasterSizer 2000. Malvern Instruments Ltd., UK).

2) Degree of dispersion (a): Standard deviation of particle diameter/average particle diameter

Meanwhile, in the cosmetic composition of the present invention, the content of the cholesteric liquid crystal compound may be preferably 0.1 wt. % to 10 wt. %, and more preferably 0.1 wt. % to 5 wt. %. When the content of a liquid crystal compound is less than 0.1 wt. %, light reflectance value is insufficient, and thus the expression of visual dependence where the color tone changes by viewing angle cannot be expected. Meanwhile, when exceeding 10 wt. %, there is a problem that the gap between the particles becomes very narrow, and thus the expression of visual dependence where the color tone changes due to angle on appearance cannot be expected.

The cosmetic composition of the present invention may further comprise a moisturizing agent, thickening agent, and other additives known in the art in a range that does not impair the effect of changing color or color tone depending on the viewing angle as described above.

Examples of the moisturizing agent may be one originated from animal oils, vegetable oils, synthetic oils, etc., and regardless of properties of sold oils, semi-solid oils, liquid oils, volatile oils, etc., the moisturizing agent may be hydrocarbons, fats and oils, hydrogenated oils, ester oils, fatty acids, lower alcohols, glycols, glycerols, higher alcohols, silicone oils, fluorine oils, lanolin derivatives, plant sterol derivatives, etc. Preferably, a polyol moisturizing agent is used alone or by two or more selected from the group consisting of glycerin, butylene glycol, propylene glycol, dipropylene glycol, diglycerin, pentylene glycol, isoprene glycol, and erythritol. The content of the moisturizing agent may be, for example, 10 wt. % to 50 wt. %.

The thickening agent is a polymer for viscosity control; is a polymer derived from plants, animals, or microorganisms; and can be used alone or by two or more selected from the group consisting of guar gum, xanthan gum, natto gum, ethyl cellulose, hydroxyethyl cellulose, carboxymethyl cellulose, sodium polyacrylate, glyceryl polyacrylate, and hydroxypropyl cellulose. The content of the thickening agent may be, for example, 0.01 wt. % to 1.0 wt. %.

Other additives include, for example, antioxidants, ultraviolet ray blocking agents/absorbers, surfactants, preservatives, diluents, pH adjusters, skin nutrients, fragrance, dyes, pigments, etc. The content of the additives can be easily selected by those skilled in the art, and may be in an amount of 0.01 wt. % to 10 wt. % based on the total weight of the composition.

The present invention provides a method for preparing a cosmetic composition comprising the cholesteric liquid crystal particle described above. Specifically, it comprises preparing a liquid crystal portion comprising a cholesteric liquid crystal; and passing the liquid crystal portion through a porous membrane and then dispersing into a continuous phase, thereby converting the liquid crystal portion to a liquid crystal particle dispersed phase, and thereby obtaining an emulsion comprising a cholesteric liquid crystal particle.

First, in the first step of the preparation method of the present invention, a liquid crystal portion comprising a cholesteric liquid crystal is prepared.

The liquid crystal portion, for example, can be prepared by using only a liquid crystal compound, when the cholesteric liquid crystal compound is in a liquid state, and even in the liquid state, when the viscosity is high, or in the solid state, it may be prepared by dissolving in an appropriate organic solvent and adjusting the viscosity. Meanwhile, the type and content of the cholesteric liquid crystal compound included in the liquid crystal portion are as described above.

The liquid crystal portion of the present invention, for example, can be prepared only with a cholesteric liquid crystal compound, and while it can be prepared by mixing with oil, when the compounding ratio exceeds 20% relative to the cholesteric liquid crystal compound, the unique structure of the liquid crystal is broken and the expected visual effect is lost, and thus the compounding ratio should be observed.

The oil may be one or more oils selected from hydrocarbon oils, ester oils, triglyceride oils, vegetable oils, and silicone oils.

In the second step, the liquid crystal portion is passed through a porous membrane and then dispersed into a continuous phase, thereby converting the liquid crystal portion to a dispersed phase in the form of a liquid crystal particle, and thereby obtaining an emulsion comprising a cholesteric liquid crystal in a particle phase.

As employed in the second step of the present invention, a method of passing a liquid crystal portion through pores of a porous membrane and then dispersing in a continuous phase uses a method known as ‘membrane emulsification.’ In the present invention, the membrane emulsification is applied, and while the liquid crystal portion passing through pores of the porous membrane is in contact with the continuous phase, the liquid crystal portion is converted into droplets between the surface of the membrane and the surface of the continuous phase, and thus the cholesteric liquid crystal particle is dispersed in the continuous phase.

The porous membrane may be, for example, porous alumina, porous zirconia, porous stainless steel, or porous glass, and preferably, the membrane of Shirasu porous glass (SPG) is used.

Shirasu porous glass membrane can be manufactured by adjusting the pore size of a membrane by 0.1 μm in a range of 0.1 μm to 50 μm in comparison with other types of membrane, and since the pores of the membrane are also easy to prepare uniformly, it is suitable for preparing the desired particle size. In addition, among organic and inorganic porous membranes that are currently produced, it has advantages that the membrane is not deformed or broken by reaction, and the surface modification is also easy to change so that it can be prepared in all of oil-in-water (01W) and water-in-oil (W/O).

Pores of the porous membrane may be selected from any of the average diameter of 0.1 μm to 2 mm, and preferably 10 μm to 50 μm.

In general, the relationship between the pore size of the membrane and the particle size to be prepared is as the following, and while there may be differences depending on the experimental condition and apparatus, the constant value (c) in the present invention was about 3 to 4.

Mathematical Formula 1

| | Membrane | Particle |
|---|---|---|
| d̅_{d} = cd̅ₚ | pore size (μm) | size (μm) |
| $( \begin{matrix}{\overline{d}}_{d} : Average particle size \\ c : Constant \\ {\overline{d}}_{p} : Average membrane pore size\end{matrix}$ | 20 30 50 | 60 ~ 80 80 ~ 120 200 ~ 250 |

In addition, there are many factors that affect the particle size such as the pressure of a dispersed phase, the stirring speed of a continuous phase, the viscosity of a dispersed phase, etc., and in order to prepare uniform particles, the pressure and stirring speed of the dispersed phase should be maintained according to the average pore size of a membrane.

$$[ Mathematical Formula 2 ] \\ {P}_{c} = \frac{4 γ cos θ}{{\overline{d}}_{p}} ( \begin{matrix}{P}_{c} : Critical Pressure & cos θ : Contact angle \\ γ : Interfacial tension & {\overline{d}}_{p} : Average pore size of membrane\end{matrix} \qquad$$

In the preparation method of the present invention, the second step may be carried out, for example, in a heated state at a temperature of 30° C. to 80° C. The heating is carried out for the purposes of securing fluidity of the liquid crystal portion, improving the solubility of other compositions added to the liquid crystal portion or the aqueous phase portion, controlling the size of the dispersed phase formed in other emulsions, etc.

Additionally, a cosmetic composition can be prepared that comprises a cholesteric liquid crystal particle having an average particle diameter of 50 μm to 500 μm, by cooling the emulsion obtained in the above step to room temperature or low temperature, for example, to −30° C.

Hereinafter, the preparation method of the cosmetic composition according to the present invention will be described with reference to the drawings.

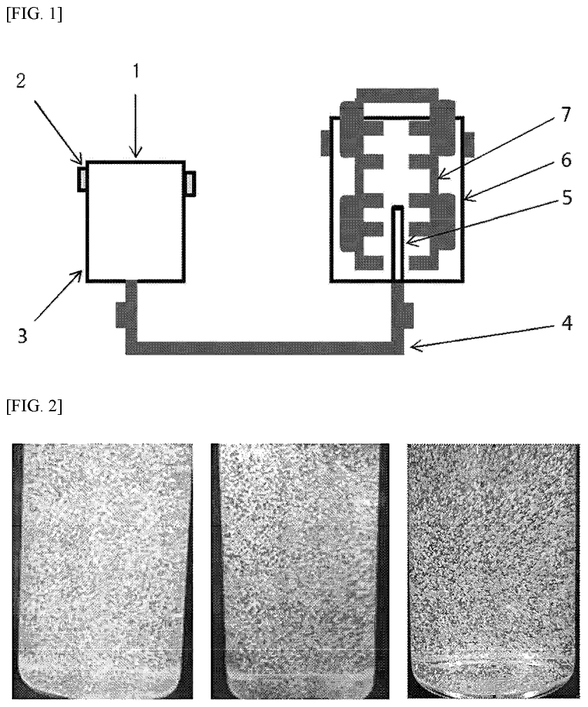

FIG. 1 is a mimetic diagram describing an apparatus to which a method for preparing cosmetics according to the present invention is applied. When FIG. 1 is referenced, first, the liquid crystal portion prepared in the first step of the preparation method of the present invention is introduced into a dispersed phase tank (3). A heating device (2) is provided in the dispersed phase tank (3), and the dispersed phase prepared in the dispersed phase tank (3), that is, the liquid crystal portion of the present invention, for example, can be heated to a temperature of 30° C. to 80° C. Meanwhile, a pressurizing device (1) is provided at one side of the dispersed tank (3), and a dispersed phase prepared in the dispersed phase tank (3) is pressurized such that the dispersed phase is to transferred to a continuous phase tank (6) through a transfer line (4). Meanwhile, a heating device (2) may be provided in the transfer line (4) and the continuous phase tank (6).

The continuous phase tank (6) is filled with a continuous phase, and for example, with an aqueous phase portion when the composition of the present invention is prepared in the form of an oil-in-water emulsion. The continuous phase may include other compositions, for example, a moisturizing agent, a thickening agent, and other additives, besides the cholesteric liquid crystal compound present in a dispersed phase in the cosmetic composition of the present invention.

The liquid crystal portion transferred through the transfer line (4) is converted into droplets in a continuous phase, by passing through a membrane module (5) comprising a porous membrane installed in the continuous phase tank (6), and is formed as a dispersed phase. The membrane module (5) comprises a porous membrane.

Meanwhile, formation of the dispersed phase can be carried out, for example, in a state where the aqueous phase portion is heated to 30° C. to 80° C. Meanwhile, a paddle (7) is provided in the continuous phase tank (6), and an emulsion comprising a cholesteric liquid crystal particle, which is present as a dispersed phase in the continuous phase of the aqueous phase portion, is stirred.

The method for preparing a cosmetic composition comprising the cholesteric liquid crystal particle of the present invention as described above has the following advantages:

1) First, it is possible to freely control the size of the cholesteric liquid crystal particle present in a dispersed phase in the aqueous phase portion, and to obtain an emulsion having a uniform particle size distribution. This is because particle size can be adjusted according to the pore size of a membrane by using a porous membrane having a uniform pore size instead of mechanical crushing, which is the principle of the conventional emulsification method.

2) Second, when the preparation method of the present invention is followed, for example, compared to other types of emulsification methods such as a stirrer-type emulsification tank and a homogenizer mixer, liquid crystal particles can be obtained in a state in which an external force applied to the produced cholesteric liquid crystal particle, for example, physical forces such as shear force due to stirring, a pressure accompanying the homogenizer, etc. is applied less. Therefore, there is little damage to liquid crystal particles.

3) As a result of 1) and 2) above, the cholestericliquid crystal's intrinsic characteristic is well expressed, and accordingly, a cosmetic composition can be prepared, in which visual dependence where color or color tone changes depending on a viewing angle is enhanced.

Hereinafter, the present invention will be described in detail with reference to the examples. The examples are intended to illustrate the present invention more specifically, and the scope of the present invention is not limited to these examples.

### Example 1: Preparation of Cosmetic Composition According to the Preparation Method of the Present Invention

In the composition shown in Table 1 below, an aqueous phase portion was introduced into the continuous phase tank (6) of FIG. 1, capable of temperature control and stirring, was heated and dissolved at 40° C. and 60 C°, and was stirred at 100 rpm to 500 rpm. Then, a cholesteric liquid crystal base (UC10, LCR Hallcrest, USA) was placed in the dispersion phase tank (3), and a pressure of 5 kPa to 40 kPa was applied to emulsify a membrane through a module (5) installed with a membrane in the aqueous phase portion, and the cholesteric liquid crystal particle was dispersed in the aqueous phase portion.

Next, the composition obtained above was cooled to 30° C. to finally prepare a cosmetic composition in which the color changes depending on a viewing angle by the cholesteric liquid crystal particles.

TABLE 1

| Classification | Component (wt. %) | Example 1 |
|---|---|---|
| Aqueous phase | Water | 86.8 |
| portion | Carbomer | 0.2 |
| | Glycerin | 5 |
| | Dipropylene glycol | 5 |
| | 1,2HEXANEDIOL | 2 |
| Cholesteric liquid | Liquid crystal base | 1 |
| crystal portion | | |

### Comparative Example 1: Preparation of Cosmetic Composition Using a Stirrer-Type Emulsification Tank

An aqueous phase portion was introduced into an emulsification tank capable of temperature control and stirring, and was heated and dissolved at 40° C. to 60° C. A cholesteric liquid crystal portion was introduced in the aqueous phase portion and stirred at 100 rpm to 500 rpm to prepare a cholesteric liquid crystal particle. In this case, the composition of the aqueous phase portion was the same as in Example 1.

Next, the cosmetic composition comprising the cholesteric liquid crystal particle obtained above was cooled to 30° C. to prepare a cosmetic composition.

### Comparative Example 2: Preparation of Cosmetic Composition Using a Homogenizer Mixer

An aqueous phase portion was introduced into an emulsification tank capable of temperature control and stirring, and was dissolved and heated at 40° C. to 60° C. A cholesteric liquid crystal portion was introduced into the aqueous phase portion and was stirred at 2,000 rpm to 3,000 rpm using a homogenizer mixer to prepare a cholesteric liquid crystal particle. In this case, the composition of the aqueous phase portion was the same as in Example 1.

Next, the cosmetic composition comprising the cholesteric liquid crystal particle obtained above was cooled to 30° C. to prepare a cosmetic composition.

FIG. 2 is an image of a cosmetic composition comprising a cholesteric liquid crystal particle prepared according to Example 1. From FIG. 2, a phenomenon can be confirmed where the cosmetic composition of the present invention exhibits a unique appearance in which color changes depending on a viewing angle by the cholesteric liquid crystal particle.

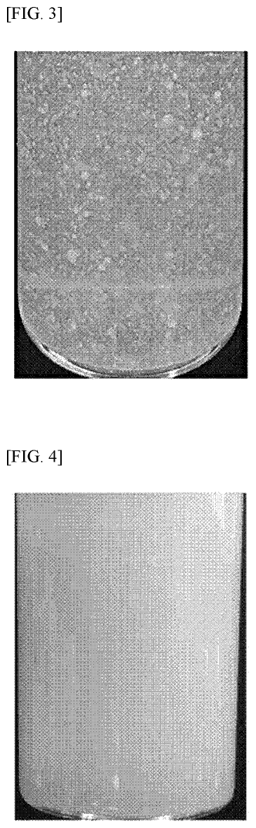

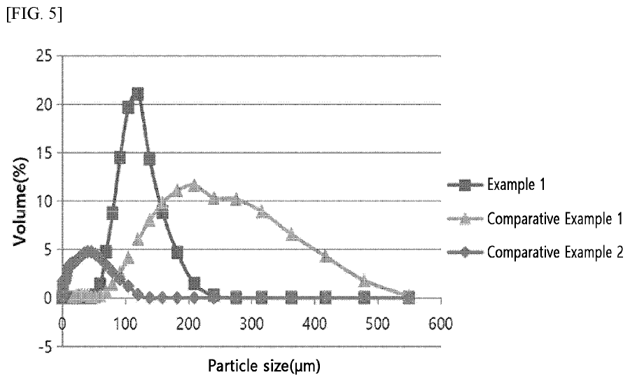

FIGS. 3 and 4 are images of the cosmetic compositions prepared according to Comparative Examples 1 and 2, respectively. From FIG. 3, in the case of the cosmetic composition prepared using a stirrer-type emulsification tank, cholesteric liquid crystal particles are made, and it has a characteristic in which the color changes depending on the viewing angle. However, since the size is not regular and the change in color is not constant, it can be confirmed that it is difficult to have a distinctive and clear appearance compared to FIG. 2. In addition, from FIG. 4, it can be confirmed that the cosmetic composition prepared using a homogenizer mixer does not show a unique characteristic that a cholesteric liquid crystal has, because the particle is too small to be observed by the naked eye and is suspended.

Table 2 shows the average particle sizes and degrees of dispersion (a) of Example 1 and Comparative Examples 1 and 2. In the research paper by R. Katoh et al. mentioned above, as a result of judging that it is considered to be uniform if a does not exceed 0.35, when prepared according to the preparation method of the present invention, the average particle size is 100 μm or more, and a is uniform to be 0.35 or less, and it is possible to observe constant changes in color depending on the angle. In the case of Comparative Example 1, the average particle size is increased, but as a is greater than 0.35 as shown in FIG. 3, it is difficult to expect constant color changes depending on the angle due to the large size difference due to uneven state of particles. When prepared by the general homogenizer preparation method of Comparative Example 2, the average particle size is 50 μm or less, and a exceeds 0.35 and is not uniform, and since the average particle size did not reach above a certain level, it can be seen to be in the form of a suspended emulsion as can be seen in FIG. 4.

TABLE 2

| | | Comparative | Comparative |
|---|---|---|---|
| | Example 1 | Example 1 | Example 2 |
| Average particle size (μm) | 116.4 | 220.07 | 25.99 |
| α | 0.27 | 0.44 | 1.03 |

### EXPLANATION OF REFERENCE NUMERALS

| | | | |
|---|---|---|---|
| 1. | Pressurizing device | 2. | Heating device |
| 3. | Dispersion phase tank | 4. | Transfer line |
| 5. | Membrane module | 6). | Continuous phase tank |
| 7. | Paddle | | |

1. A cosmetic composition, comprising a cholesteric liquid crystal particle having an average diameter of 50 μm to 500 μm.

2. The cosmetic composition of claim 1, wherein the cholesteric liquid crystal particle is present in the form of a dispersed phase or continuous phase of an oil-in-water emulsion or water-in-oil emulsion.

3. The cosmetic composition of claim 1, wherein the degree of dispersion (α) of the cholesteric liquid crystal particle is 0.35 or less.

4. The cosmetic composition of claim 1, wherein the weight of the cholesteric liquid crystal particle is 0.1 wt. % to 10 wt. % based on the total weight of the composition.

5. The cosmetic composition of claim 2, wherein the emulsion is an oil-in-water emulsion, and an aqueous portion of the oil-in-water emulsion comprises 10 wt. % to 50 wt. % of a moisturizing agent, 0.01 wt. % to 1.0 wt. % of a thickening agent, and a residual amount of purified water based on the total weight of the composition

6. The cosmetic composition of claim 5, wherein the moisturizing agent is one or two or more selected from the group consisting of glycerin, butylene glycol, propylene glycol, dipropylene glycol, diglycerin, pentylene glycol, isoprene glycol, and erythritol.

7. The cosmetic composition of claim 5, wherein the thickening agent is one or two or more selected from the group consisting of guar gum, xanthan gum, natto gum, ethyl cellulose, hydroxyethyl cellulose, carboxymethyl cellulose, sodium polyacrylate, glyceryl polyacrylate, and hydroxypropyl cellulose.

8. A method for preparing a cosmetic composition, comprising:
preparing a liquid crystal portion comprising a cholesteric liquid crystal; and
passing the liquid crystal portion through a porous membrane and then dispersing into a continuous phase, thereby converting the liquid crystal portion to a liquid crystal particle dispersed phase, and thereby obtaining an emulsion comprising a cholesteric liquid crystal particle.

9. The method of claim 8, further comprising cooling the emulsion.

10. The method of claim 8, wherein the porous membrane is porous alumina, porous zirconia, porous stainless steel, or porous glass.

11. The method of claim 8, wherein pores of the porous membrane have an average diameter of 0.1 μm to 2 mm.
